(19) United States
(12) Patent Application Publication
(10) Pub. No.: US 2008/0152671 A1
(43) Pub. Date: Jun. 26, 2008

(54) NOVEL VACCINATION

(75) Inventor: Martine J. Denis, Rixensart (BE)

Correspondence Address: GLAXOSMITHKLINE;Corporate Intellectual Property - UW2220, P.O. Box 1539, King of Prussia, PA, 19406-0939, US

(73) Assignee: GlaxoSmithKline Biologicals S.A.

(21) Appl. No.: 12/044,209

(22) Filed: Mar. 7, 2008

Related U.S. Application Data
(62) Division of application No. 10/923,407, filed on Aug. 20, 2004.
Application No. 10/923,407 is a continuation-in-part of application No. PCT/EP03/04855, filed on May 7, 2003.

(30) Foreign Application Priority Data
May 9, 2002 (GB) 0210682.1

(51) Int. Cl. A61K 39/00; A61P 37/00

(52) U.S. Cl. 424/204.1

(57) ABSTRACT

The invention relates to the use of an EBV membrane antigen or derivative thereof in combination with a suitable adjuvant in the manufacture of a vaccine for the prevention of infectious mononucleosis (IM), and to vaccine compositions suitable for prevention of IM.

### CROSS REFERENCE TO RELATED APPLICATIONS

This application is a divisional of U.S. application Ser. No. 10/923,407, filed 20 Aug. 2004, which is a continuation-in-part of international application PCT/EP03/04855, filed 7 May 2003, the disclosures of which are incorporated herein by reference. This application also claims benefit of the earlier filing date of United Kingdom Application 0210682.1, filed 9 May 2002.

### FIELD OF INVENTION

This invention relates to prevention of Epstein-Barr virus (EBV) infection, more specifically to the prevention of infectious mononucleosis resulting from EBV infection. More specifically the invention relates to the use of EBV antigens, in particular the glycoprotein known as gp350 and derivatives thereof, in vaccines to prevent EBV infection and/or infectious mononucleosis.

### BACKGROUND OF INVENTION

EBV is a member of the herpesvirus group and an important pathogen. It causes infectious mononucleosis (IM) in humans, a disease also termed glandular fever. In a recent report, direct health costs associated with infectious mononucleosis in the United States were estimated to reach as much as 16 million US dollars per year. Moreover the disease causes significant indirect costs related to the long periods of absence from work/school that frequently accompany infectious mononucleosis.

The virus is also associated with a wide variety of other clinical conditions, many of which are malignant. These include Burkitt's Lymphoma, B cell lymphomas and smooth muscle tumours in immunosuppressed patients, some T-cell lymphomas, Hodgkin's disease, X-linked lymphoproliferative syndrome, nasopharyngeal carcinoma, gastric carcinoma and oral hairy leukoplakia.

In Western communities about 85-90% of all adults carry the EBV virus. In developing countries the infection level approaches 100% by the age of two. Natural primary infection occurs during childhood and is generally asymptomatic. In common with other herpesviruses, EBV establishes a persistent infection which is maintained lifelong.

In developed countries primary infection is often delayed for several years. Following first time infection during adolescence or young adulthood, clinical infectious mononucleosis develops in about half of the instances. In the United States alone, it is estimated that there are more than 100,000 new cases per year. Therefore even ignoring the virus' association with various human cancers, EBV is an important target for a vaccine. Yet no vaccine so far exists.

An attenuated virus approach to a vaccine for EBV has found little favour due to the possibility that the viral DNA may prove oncogenic. Most of the approaches to the development of an EBV vaccine have therefore concentrated on the virus membrane antigen which consists of at least three glycoproteins of molecular weights about 350,000 daltons (gp350), about 220,000 daltons (gp220) and about 85,000 daltons (gp85). In the literature gp350 and gp220 are referred to using a variety of molecular weight ranges e.g. gp340 or gp300 for gp350, and gp200 for gp220. Herein the glycoproteins are referred to as gp350 and gp220 are are collectively referred to as gp350/220 protein(s).

An alternatively spliced, single gene encodes the gp350/220 proteins and results in the generation of gp350 and gp220 mRNA transcripts. The gene produces two expression products, the gp350 and gp220 proteins. The open reading frame for the gp350/220 DNA sequence is 2721 bp and the entire reading frame encodes the 907 amino acids of gp350 (see U.S. Pat. No. 4,707,358 issued to Kieff 1987). The spliced version of the reading frame covers 2130 bases and translates into gp220 protein, a 710 amino acid sequence.

Recombinant production of these proteins frequently resulted in a mixture of gp350 and gp220 protein being produced. Modified versions of the EBV gp350/220 proteins are also known in the art, for example recombinant truncated constructs of the gp350/220 gene lacking the membrane spanning sequence. Such constructs still produce a mixture of the two gp350 and gp220 but deletion of the membrane spanning region permits secretion of the proteins.

Partially purified preparations of gp350/220 have been described as early as the seventies, but they remained poorly characterized and they were not all immunogenic (Boone CW et al, J Natl Cancer Inst (1973) 50:841). Later on, highly purified preparations of antigenically active gp350 protein from native and recombinant sources have been obtained (Morgan A J, North J R and Epstein M A. Purification and properties of the gp340 component of Epstein-Barr Virus membrane antigen in an immunogenic form. J. Gen. Virol. (1983) 64: 455-460; Thorley-Lawson D and Poodry C A. Identification and isolation of the main component (gp350-gp220) of Epstein-Barr Virus responsible for generating neutralizing antibodies in vivo. J. Virol. (1982) 43: 730-736; Emini E A, Schleif W A, Armstrong M E, Silberklang M, et al Virol (1988) 166:387-393; Madej M, Conway M J, Morgan A J et al Vaccine (1992) 10:777-782). However, many of these purification methods for purifying gp350/220 are not compatible with manufacturing of a commercial vaccine (e.g. lack of purity, unsufficient yield).

EP 0 769 056 describes non-splicing variants of the EBV gp350/220 DNA sequence which allows production of homogeneous recombinant gp350, that is, production of recombinant gp350 independently of gp220. This is achieved by the removal of some or all of the RNA splice site signals in the gp350 gene and expression of the gene in a suitable host cell. Preferably but not necessarily the EBV antigen is homogeneous gp350 without significant gp220 present.

Several publications reviewed the rationale and strategies for EBV vaccine development. Arrand (1992) in The Cancer Journal, 5 (4) “Prospects for a Vaccine against Epstein-Barr virus” stated that recent results in model systems were promising. Arrand was optimistic about the prospects for an EBV vaccine coming into clinical use. However, ten years on from there, no EBV vaccine is anywhere near becoming a reality. The consensus from the published literature seems to be that EBV prevents a particular challenge for vaccination because of the prevalence of the virus and the number of diseases with which it is associated.

The preclinical models that have lead some authors like Arrand to suggest the feasibility of an EBV vaccine were reviewed by Khanna et al (1999) Immunol Rev 170, 49-64. Several primate and/or rodent models exist for EBV infection. The protective efficacy of different EBV vaccines has been assessed in some of them—with variable level of success. Most work in this regard has been concentrated on the cotton-top tamarin (Saguinus oedipus oedipus), in which multiple B-cell lymphomas develop following inoculation with high-titred EBV. The owl monkey (Aotus trivirgatus) is susceptible to EBV-induced lymphoma, while the common marmoset (Callithrix jaccus) develops a transient increase in lymphocyte counts following EBV inoculation. Shedding of EBV in the oral cavity can also be observed in the common marmoset (Cox et al (1996) J Gen Virol 77, 1173-1180). The available murine model consists of injecting peripheral blood mononuclear cells from an EBV-seropositive donor into SCID mice, which results in the development of a B-cell lymphoma.

One human trial was reported in 1995 that used a live recombinant vaccinia strain expressing gp220/350 under the 11K vaccinia promoter and claimed marginal success. The construct was tested in EBV-positive and vaccinia virus-exposed adults, EBV-positive, non vaccinia-exposed juveniles, and EBV and vaccinia virus-naïve infants in children (Gu et al (1995) Dev Biol Stand. Basel, Karger, 84, 171-177). However, no study was made of EBV linked disease and no inference could be drawn in relation to IM which does not affect infants and children.

### SUMMARY OF INVENTION

Surprising results have now been found in a human vaccination trial using a subunit vaccine comprising EBV membrane antigen in combination with a suitable adjuvant. This vaccine is effective to prevent IM in a population of adolescents and young adults. These results were not expected or predictable.

In none of the experimental models of EBV infection described previously could the specific symptoms of infectious mononucleosis (IM) be observed, e.g. intense fatigue, lymphadenopathy, fever. Moreover, a likely protective mechanism of an IM vaccine relates to induction of mucosal antibodies and blocking the spread of EBV from the oro-pharynx (where infection first takes place in humans) to the peripheral blood. None of the animal models described, which use parenteral (most often intraperitoneal) challenge and/or monitoring of virus persistence in the oro-pharynx, can evaluate the ability of the vaccine to block such spread of the virus. Furthermore, the single human experiment described in the literature was not relevant to infectious mononucleosis.

### BRIEF DESCRIPTION OF DRAWINGS

FIG. 1. Timing of occurrence of infectious mononucleosis in vaccines and placebo recipients

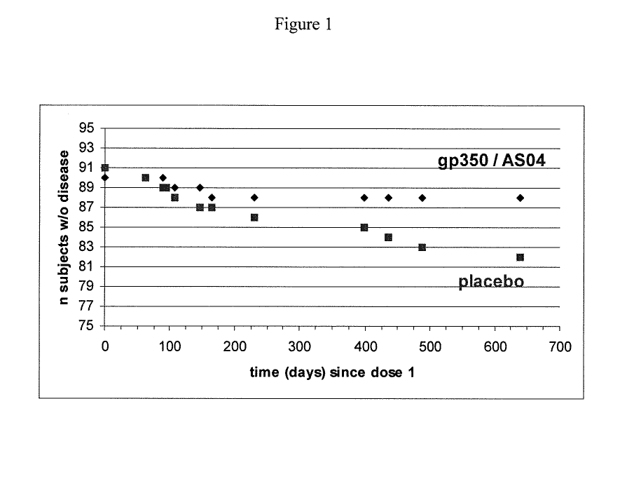

### DETAILED DESCRIPTION OF INVENTION

In a first aspect the invention provides the use of an EBV membrane antigen or derivative thereof in combination with a suitable adjuvant in the manufacture of a vaccine for the prevention of infectious mononucleosis (IM).

The invention is particularly useful in a population of adolescents and young adults in the age range 11 to 25, which is the age range most susceptible to IM.

Preferably the EBV antigen is gp350 or gp220 or a derivative thereof. Derivatives include truncates, peptides and other modified versions of gp350/220 such as those disclosed herein or known in the art. Such derivatives include peptides having at least contiguous amino acids of the linear sequence of gp350 or 220 which are recognised by EBV-neutralising antibodies and/or bind to human CD21 (also referred to as CR2). Preferred derivatives include at least one of residues 21-24 or 25-27 of gp350/220.

Particularly preferred for use in the invention is a homogeneous preparation of gp350, which means a preparation of gp350 which is uncontaminated or substantially uncontaminated with gp220. Such a preparation may be obtained for example by production of recombinant gp350 as described in EP 0 769 056.

Suitable adjuvants for use in the invention include mineral salts such as an aluminium or calcium salts, in particular aluminium hydroxide, aluminium phosphate and calcium phosphate.

Preferably the adjuvant further comprises a non-toxic bacterial lipopolysaccharide derivative such as 3 De-O-acylated monophosphoryl lipid A (3D-MPL).

In another aspect the invention provides a vaccine composition comprising a homogeneous preparation of EBV gp350, a mineral salt such as an aluminium or calcium salt and a non-toxic bacterial lipopolysaccharide derivative such as 3D-MPL.

A particularly preferred vaccine according to this aspect of the invention comprises a homogeneous preparation of gp350 in combination with aluminium hydroxide and 3D-MPL.

Most preferably the gp350 is recombinant gp350 prepared from a non-splicing variant of the DNA expressing the gp350/220 proteins such as that described in EP 0 769 056.

Another preferred adjuvant comprises a saponin such as QS21 which is an Hplc purified non-toxic fraction derived from the bark of Quillaja Saponaria Molina. Optionally this may be admixed with 3D-MPL, optionally together with a carrier.

The method of production of QS21 is disclosed in U.S. Pat. No. 5,057,540.

Non-reactogenic adjuvant formulations containing QS21 are described in WO 96/33739. Such formulations comprising QS21 and cholesterol have been shown to be successful TH1 stimulating adjuvants when formulated together with an antigen. Thus the present invention may employ an adjuvant comprising a combination of QS21 and cholesterol.

Further suitable adjuvants for use in the invention include immunomodulatory oligonucleotides, for example unmethylated CpG sequences as disclosed in WO 96/02555.

Combinations of different adjuvants, such as those mentioned hereinabove, are also contemplated for use in the vaccine compositions described herein. For example, as already noted, QS21 can be formulated together with 3D-MPL. The ratio of QS21: 3D-MPL will typically be in the order of 1:10 to 10:1; preferably 1:5 to 5:1 and often substantially 1:1. The preferred range for optimal synergy is 2.5:1 to 1:13D-MPL: QS21.

Preferably a carrier is also present in the vaccine composition according to the invention. The carrier may be an oil in water emulsion, or a mineral salt such as a calcium or aluminium salt, for example calcium phosphate, aluminium phosphate or aluminium hydroxide.

A preferred oil-in-water emulsion comprises a metabolisable oil such as squalene, alpha tocopherol or Tween 80. Additionally the oil in water emulsion may contain span 85 and/or lecithin and/or tricaprylin.

Typically for human administration QS21 and 3D-MPL will be present in a vaccine in the range of 1 μg-200 μg, such as 10-100 μg, preferably 10 μg-50 μg per dose. Typically the oil in water will comprise from 2 to 10% squalene, from 2 to 10% alpha tocopherol and from 0.3 to 3% tween 80. Preferably the ratio of squalene: alpha tocopherol is equal to or less than 1 as this provides a more stable emulsion. Span 85 may also be present at a level of 1%. In some cases it may be advantageous that the vaccines of the present invention will further contain a stabiliser.

Non-toxic oil in water emulsions preferably contain a non-toxic oil, e.g. squalane or squalene, an emulsifier, e.g. Tween 80, in an aqueous carrier. The aqueous carrier may be, for example, phosphate buffered saline.

A particularly potent adjuvant formulation involving QS21, 3D-MPL and tocopherol in an oil in water emulsion is described in WO 95/17210.

Enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p407-419) and has the following structure:

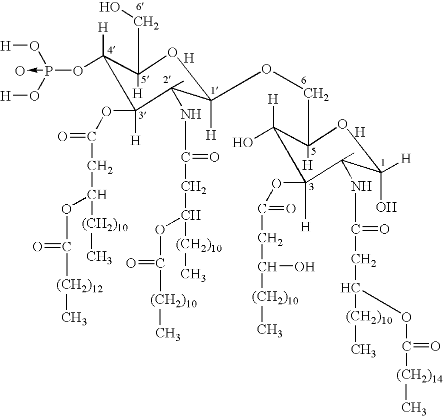

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. A preferred form of 3D-MPL is in the form of an emulsion having a small particle size less than 0.2 μm in diameter, and its method of manufacture is disclosed in WO 94/21292. Preferably, the particles of 3D-MPL are small enough to be sterile filtered through a 0.22 micron membrane (as described in European Patent number 0 689 454).

Examples of such derivatives of LPS are described below.

The bacterial lipopolysaccharide derived adjuvants to be formulated in the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (U.S. Pat. No. 6,005,099 and EP 0 729 473 B1; Hilgers et al., 1986, Int.Arch.Allergy.Immunol., 79(4):392-6; Hilgers et al., 1987, Immunology, 60(1):141-6; and EP 0 549 074 B1). Particularly preferred bacterial lipopolysaccharide adjuvants are 3D-MPL and the β(1-6) glucosamine disaccharides described in U.S. Pat. No. 6,005,099 and EP 0 729 473 B1.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

A preferred disaccharide adjuvant for combination with CpG, is a purified or synthetic lipid A of the following formula:

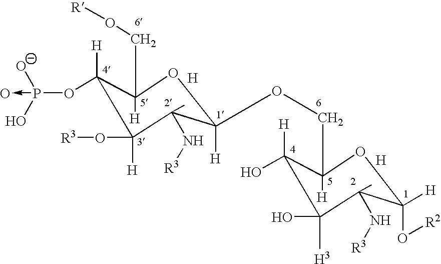

wherein R2 may be H or PO3H2; R3 may be an acyl chain or 13-hydroxymyristoyl or a 3-acyloxyacyl residue having the formula:

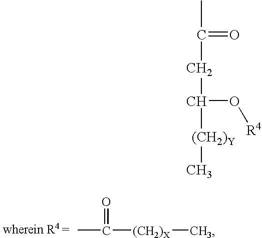

and wherein X and Y have a value of from 0 up to about 20.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

The vaccine of the present invention will contain an immunoprotective quantity of the antigen and may be prepared by conventional techniques.

Vaccine preparation is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in each vaccine dose is selected as an amount which induces an immunoprotective response without significant, adverse side effects in typical vaccinees. Immunoprotective in this context does not necessarily mean completely protective against infection; it means protection against disease associated with the virus, i.e. infectious mononucleosis. The amount of antigen will vary depending upon which specific immunogen is employed. Generally, it is expected that each dose will comprise 1-1000 μg of protein, preferably 2-100 μg, most preferably 10-50 μg. An optimal amount for a particular vaccine can be ascertained by standard studies involving observation of antibody titres and other responses in subjects. Following an initial vaccination, subjects may receive a boost in about 4 weeks.

### EXAMPLES

### Example 1

### Materials and Methods

### Study Population

This first study was performed at the University of Liege, Belgium, in 67 healthy volunteers aged 18-25, either positive or negative for serological markers of EBV infection, and therefore respectively not at risk and at risk of EBV infection and infectious mononucleosis. Local ethics committee approval from the study centre and written informed consent for each subject were obtained. Women of child-bearing age agreed to use appropriate contraception during the first 7 months of the study.

Exclusion criteria included clinical signs of acute illness at time of study entry, history of infectious mononucleosis, major congenital defects or serious chronic illness, any chronic treatment with immunosuppressive drugs including corticosteroids, any immunosuppressive or immunodeficient condition, history of chronic alcohol consumption and/or intravenous drug abuse, any history of sensitivity to vaccine components, simultaneous participation in any other clinical trial, pregnancy or lactation, simultaneous administration of any other vaccine(s), administration of immunoglobulins during the study or within three months preceding the first vaccine dose.

### Vaccines

The vaccines were formulated by GlaxoSmithKline Biologicals (Rixensart, Belgium). Each 0.5 ml dose of the gp350 vaccine, in monodose vials, contained 50 μg of gp350 either adsorbed onto 0.5 mg aluminium hydroxide (Al(OH)₃) or formulated with 0.5 mg aluminium hydroxide and 50 μg 3D-MPL. Gp350 was produced in Chinese Hamster Ovary cells as a truncated product expressed in the culture medium, and in a mutated form allowing for production of gp350 in the absence of the gp220 isoform (as described in EP 769 056, Jackman et al). Culture of the adherent producing cell line was made in the presence of foetal bovine serum. Gp350 was then purified from the culture supernatant according to the methods described by Jackman et al.

### Study Design

The study was a double-blind, randomised study in which the two vaccine formulations were administered according to a 0-1-6 months schedule. Subjects recorded solicited and unsolicited signs and symptoms on diary cards following each vaccine dose until day 7, with subject follow-up for adverse events until day 28. Blood samples were drawn on months 0, 1, 2, 6 and 7 for determination of anti-gp350 antibody titres as well as anti-EBV antibodies (antibodies to non-vaccine antigens, marker of EBV infection). An additional visit was organised at year 3. The subjects were then interviewed for the occurrence of infectious mononucleosis over the 3 year period since their previous visit, and blood samples were drawn for determination of anti-gp350 immunity and EBV infection status.

### Immunological Testing

All samples were analysed in a blinded fashion. Anti-gp350 antibodies were determined using an ELISA developed at GlaxoSmithKline Biologicals. Anti-EBV (non-vaccine antigens) antibodies were determined using a commercial ELISA kit specific for Viral Capsid Antigens, and confirmed by immunofluorescence assay. Discrepancies between ELISA and immunofluorescence were solved by re-test and further EBV testing using a panel of immunofluorescence and ELISA assays (anti-VCA, -EA, -EBNA, -p107) at an external laboratory (Swedish Institute for Infectious Disease Control, Stockholm, Sweden). The neutralizing titre of the sera was measured by their ability to inhibit immortalization of fresh human B lymphocytes by EBV. Similarly, EBV-specific cell-mediated immunity was assessed by the ability of the subjects' lymphocytes to inhibit immortalization upon EBV infection (outgrowth inhibition assay).

### Statistical Methods:

Binomial tests were used to compare incidences of cases in vaccinated EBV-seronegative subjects to expected incidences in unvaccinated seronegative subjects of similar age. Calculations were performed using Unistat Statistical Package (Unistat Ltd, England).

### Results and Discussion

### EBV Infection Cases

A total of 17 subjects, either vaccinated with the gp350/Alum or gp350/Alum+3D-MPL and gp350 formulation, who were seronegative for markers of EBV infection at month 7, participated in the year 3 visit. Nine of them had received the Alum formulated vaccine, and eight of them the Alum+3D-MPL formulation. All of them were evaluated for EBV infection over the 3 year period between the 2 study visits. Results are presented in Table 1 below.

TABLE 1
EBV infection cases in vaccinated subjects of study n°1

| | | No. EBV infected | % infected at yr. 3 (95% |
|---|---|---|---|
| Vaccine received | No. | at yr. 3 | confidence interval) |
| Gp350/Alum | 9 | 3 | 33.3% (7.49-70.07) |
| Gp350/ | 8 | 1 | 12.5% (0.32-52.65) |
| Alum + 3D-MPL | | | |
| None | 17 | 4 | 23.5% (6.81-49.90) |

In total, 4 of the 17 vaccinated subjects were infected by EBV over the 3-year period. More precisely, a proportion of 4 cases was observed for a total follow-up of 636 months×subjects. All vaccinated subjects had gp350 antibodies at month 7, and therefore these data show that EBV infections occur in vaccinated subjects despite induction of anti-gp350 immunity.

The annual attack rate of EBV infection in a population of unvaccinated EBV-seronegative young adults is reported to reach at least 12% (reviewed by Evans and Niederman, Epstein-Barr virus, in Viral Infections of Humans, epidemiology and control. 1991: pp 265-292. Plenum medical book company). To obtain a more accurate estimate of this attack rate, a sero-epidemiological study was also conducted in Belgium, among the same University population that participated in the clinical trials. More than 800 serum samples from subjects aged 17 to 46 years were obtained and their EBV serological status determined by anti-VCA ELISA testing and immunofluorescence. The data were then computed to model the proportion of EBV-seronegative subjects according to age. A constant rate of EBV infection in seronegative adolescents/young adults was assumed, and therefore an exponential regression curve was calculated to fit the data. The following formula was obtained:

y=161.14 e^{-1269x}

with y=percentage seronegative subjects at age x, and x=the age in years. The formula allowed calculation of an estimated annual attack rate of EBV infection in our population of adolescents/young adults at risk, which was found equal to 11.9% and rounded to 12%.

Twelve percent annual attack rate of EBV infection would correspond to one expected infection case per 100 months×subjects of follow-up in seronegative unvaccinated subjects. No significant difference was seen between the observed proportion of cases in vaccinated subjects of study n^{o} 1 (4/636 months×subjects) and expected proportion in unvaccinated subjects (1/100 months×subjects). The data therefore suggest a lack of protection against EBV infection in our study population vaccinated with either gp350/Alum or gp350/Alum+3D-MPL.

### Infectious Mononucleosis Cases

As mentioned above, a total of 17 subjects, either vaccinated with the gp350/Alum or gp350/Alum+3D-MPL formulation, who were seronegative for markers of EBV infection at month 7, participated in the year 3 visit. Only one of them reported symptoms consistent with infectious mononucleosis (at month 38, fever, malaise/fatigue, pharyngitis, lymph hyperplasia, of a duration ranging between one week and one month). However, the immunological profile of the subject did not confirm the occurrence of EBV-related infectious mononucleosis (Viral Capsid Antigens IgG and IgM ELISAs negative, EBV immunofluorescence negative, gp350 ELISAs negative, EBV neutralization negative and EBV cell-mediated immunity negative). It was therefore concluded that none of the 17 subjects had developed infectious mononucleosis resulting from EBV infection over the 3 year period. More precisely, no case of infectious mononucleosis was detected for a total follow-up of 636 subjects×months.

Very few studies were conducted to define the annual attack rate of infectious mononucleosis in a population of unvaccinated EBV-seronegative young adults. From the available epidemiological data (reviewed by Evans and Niederman, 1993; and D. Crawford personal communication), a 7 percent annual incidence of infectious mononucleosis can be expected in a population of susceptible subjects. But the published data were obtained from surveys conducted in the United States and United Kingdom only. A retrospective epidemiological survey was then initiated to evaluate the incidence of the disease in Belgium. Questionnaires were distributed to 3 different populations (staff of GlaxoSmithKline Biologicals, students of the Faculty of Veterinary Medicine of the University of Liege and selected classes from French-speaking Brussels' schools). Three thousand five hundred and ninety seven replies were obtained. These corresponded to subjects aged 13 to 66 years (average 27.9 years), with a male to female ratio of 0.82. Almost one fifth of them reported a history of infectious mononucleosis. To determine the corresponding incidence of the disease in seronegative subjects, the formula calculated from the sero-epidemiological study described above was used. For each year of age between 16 and 25, the estimated number of seronegative subjects was obtained by multiplying the number of replies from subjects of that age or older by the percentage seronegative subjects at that age (obtained from the formula). The percentage infectious mononucleosis cases reported at that age was then calculated. Overall, between 16 and 25 years of age, the calculated annual incidence of infectious mononucleosis in seronegative subjects was found to reach 9.2 percent in the Belgian population surveyed. Approximately 10% of these cases can be considered as related to Cytomegalovirus rather than Epstein-Barr infection, and therefore an 8 percent annual incidence of EBV-related infectious mononucleosis can be expected to occur in an unvaccinated population comparable to that participating to study n^{o} 1.

Eight percent annual incidence of disease correspond to one expected infectious mononucleosis case per 150 months×subjects of follow-up in unvaccinated subjects. The difference between the observed proportion of cases in vaccinated subjects (0/17 subjects) and expected number in unvaccinated subjects (1/150 months×subjects, which would correspond to 4 cases in our trial) was relatively close to statistical significance (p<0.12). The data therefore suggest the efficacy of the vaccine to protect against infectious mononucleosis. In combination with the results from Example 2 (see below), thus producing a bigger and more realistic population sample on which to perform statistical analysis, the results are found to be statistically significant

### Example 2

### Materials and Methods

### Study Population

The second study was performed in two centres, the University of Liege and Catholic University of Louvain, Belgium, in 81 healthy volunteers aged 18-45, all negative for serological markers of EBV infection, and therefore at risk of EBV infection and infectious mononucleosis. Local ethics committee approval and written informed consent for each subject were obtained. Women of child-bearing age agreed to use appropriate contraception for the duration of the study.

Exclusion criteria included clinical signs of acute illness at time of study entry, history of infectious mononucleosis, major congenital defects or serious chronic illness, any chronic treatment with immunosuppressive drugs including corticosteroids, any immunosuppressive or immunodeficient condition, history of chronic alcohol consumption and/or intravenous drug abuse, any history of sensitivity to vaccine components, simultaneous participation in any other clinical trial, pregnancy or lactation, simultaneous administration of any other vaccine(s), administration of immunoglobulins during the study or within three months preceding the first vaccine dose.

### Vaccines

Three different vaccine formulations were evaluated in study n^{o} 2. Each 0.5 ml dose of the gp350 vaccine, in monodose vials, contained 50 μg of gp350 either unadjuvanted, adsorbed onto 0.5 mg aluminium hydroxide (Al(OH)₃) or formulated with 0.5 mg aluminium hydroxide and 50 μg 3D-MPL. Gp350 was produced in Chinese Hamster Ovary cells as a truncated product expressed in the culture medium, and in a mutated form allowing for production of gp350 in the absence of the gp220 isoform (see EP 769 056, Jackman et al). Culture of the producing cell line was made in suspension in serum-free medium. Gp350 was then purified from the culture supernatant.

### Study Design

The study was a double-blind, randomised study in which the three vaccine formulations were administered according to a 0-1-6 months schedule. Subjects recorded solicited and unsolicited signs and symptoms on diary cards following each vaccine dose until day 7, with subject follow-up for adverse events until day 28. Blood samples were drawn on months 0, 1, 2, 6 and 7 for determination of anti-gp350 antibody titres as well as anti-EBV antibodies (antibodies to non-vaccine antigens, marker of EBV infection).

### Immunological Testing

All samples were analysed in a blinded fashion. Anti-gp350 antibodies were determined using an in house ELISA. Anti-EBV (non-vaccine antigens) antibodies were determined using a commercial ELISA kit specific for Viral Capsid Antigens, and confirmed by immunofluorescence assay. The neutralizing titre of the sera was measured by their ability to inhibit immortalization of fresh human B lymphocytes by EBV. Similarly, EBV-specific cell-mediated immunity was assessed by the ability of the subjects' lymphocytes to inhibit immortalization upon EBV infection (outgrowth inhibition assay).

### Statistical Methods:

Binomial tests were used to compare incidences of cases in vaccinated EBV-seronegative subjects to expected incidences in unvaccinated seronegative subjects of similar age. Calculations were performed using Unistat Statistical Package (Unistat Ltd, England). Results and discussion:

### EBV Infection Cases

The number of EBV infection cases detected over the 7 months duration of study n^{o} 2 is presented in Table 2 below.

TABLE 2
EBV infection cases during study n° 2

| | | No. EBV | % EBV | 95% confidence |
|---|---|---|---|---|
| Vaccine received | No. | infected | infected | interval |
| Gp350/Alum | 27 | 3 | 11.11 | 2.35-29.16 |
| Gp350/Alum + 3D-MPL | 27 | 2 | 7.41 | 0.91-24.29 |
| Gp350 | 27 | 1 | 3.70 | 0.09-18.97 |
| None | 81 | 6 | 7.41 | 2.77-15.54 |

At month 7, 6 of the 81 vaccinated subjects had seroconverted. More precisely, a proportion of 6 cases was observed for a total follow-up of 551 months×subjects. Five of these 6 infection cases occurred more than 1 month after the second vaccine injection, at a time when all but two subjects (from the group immunized with the unadjuvanted vaccine) had developed antibodies to gp350, and thereby when protection was expected to be induced.

As mentioned above, the expected annual attack rate of EBV infection in a population of unvaccinated EBV-seronegative young adults is at least 12%. This would correspond to one expected infection case per 100 months×subjects of follow-up in unvaccinated subjects. No difference was seen between the observed proportion of cases in vaccinated subjects (6/81 subjects or 6/551 months×subjects) and expected proportion in unvaccinated subjects (1/100 months×subjects). The data therefore do not support protection against EBV infection in our study population vaccinated with either gp350/Alum, gp350/Alum+3D-MPL or gp350 alone.

### Infectious Mononucleosis Cases

As mentioned above, a total of 81 subjects was either vaccinated with gp350/Alum, gp350/Alum+3D-MPL or gp350 alone. None of these subjects reported symptoms consistent with infectious mononucleosis during the trial. In other words, no case of infectious mononucleosis was detected for a total follow-up of 551 subjects×months. As mentioned above, the expected annual attack rate of infectious mononucleosis in a population of unvaccinated EBV-seronegative young adults in Belgium equals 8 percent or one infectious mononucleosis case per 150 months×subjects of follow-up in unvaccinated subjects. The difference between the observed proportion of cases in vaccinated subjects (0/81 subjects) and expected proportion in unvaccinated subjects (1/150 months×subjects=4 cases for 81 subjects over 7 months) was clear, although it did not quite reach statistical significance (p<0.2). The lack of statistical power of the study design (relatively small number of subjects and very short duration of follow-up) prevented any statistical conclusion on the difference in proportion of cases. But the data are nevertheless considered as supportive of the efficacy of the vaccine to protect against infectious mononucleosis.

Pooling of Data from Examples 1 and 2

By combining the results of the EBV vaccinated subjects of studies 1 and 2, a total of 98 subjects were followed for an average duration of 12 months, and none of them developed infectious mononucleosis. This FIGURE is significantly different from the expected annual incidence of 8 percent disease in unvaccinated seronegative subjects (p<0.02). And therefore we can conclude in the efficacy of the EBV gp350 vaccine in preventing infectious mononucleosis in adolescents/young adults at risk.

### Example 3

### Materials and Methods

### Study Population

The multicentric study was conducted in Belgium, at 6 different locations (Antwerp, Brussels, Charleroi, Leuven, Liège and Woluwe). It enrolled a total of 183 healthy volunteers aged 16-25 years, negative for serological markers of EBV infection (as confirmed by EBV immunofluorescence testing), and therefore at risk of EBV infection and infectious mononucleosis. Local ethics committee approval from the study centers and written informed consent for each subject were obtained. Female volunteers agreed to use appropriate contraception during the first 7 months of the study, and urine pregnancy tests were performed prior to each vaccine injection.

Exclusion criteria included clinical signs of acute illness at time of study entry, fever, history of infectious mononucleosis, major congenital defects or serious chronic illness, history of any neurological disorders or seizures, any chronic treatment with immunosuppressive drugs including corticosteroids, any immunosuppressive or immunodeficient condition, history of intravenous drug abuse, any history of sensitivity to vaccine components, simultaneous participation in any other clinical trial, pregnancy or lactation, simultaneous administration of any other vaccine(s), administration of immunoglobulins during the study or within three months preceding the first vaccine dose.

### Vaccines

The vaccines were formulated by GlaxoSmithKline Biologicals (Rixensart, Belgium). Each 0.5 ml dose of the gp350 vaccine, in monodose vial, contained 50 μg of gp350 formulated with 0.5 mg aluminium hydroxide and 50 μg 3D-MPL. Gp350 was produced in Chinese Hamster Ovary cells as a truncated product expressed in the culture medium, and in a mutated form allowing for production of gp350 in the absence of the gp220 isoform (as described in EP 769 056, Jackman et al). Culture of the producing cell line was made in suspension, in the absence of foetal bovine serum. Gp350 was then purified from the culture supernatant.

The 0.5 ml dose of placebo, also in monodose vial, contained 0.5 mg aluminium hydroxide.

### Study Design

The study was a double-blind, randomised study in which the vaccine and placebo were administered intramuscularly into the deltoid according to a 0-1-5 months schedule. Blood samples were drawn on months 0, 1, 5, 6 and 19 for determination of anti-EBV antibodies (antibodies to non-vaccine antigens, marker of EBV infection). During the first study visit, subjects were trained to recognise symptoms of infectious mononucleosis, and were requested to contact the investigator each time they suspected they contracted the disease. Subjects were also contacted by the center on a monthly basis and asked if they experienced symptoms of infectious mononucleosis. For each suspicion of infectious mononucleosis, an additional visit was organised where the symptoms of disease and their date of onset were recorded, and blood samples were collected for laboratory analysis (see below). When infectious mononucleosis was confirmed, further extra visits were then organised on a monthly basis until the subject recovered.

### Infectious Mononucleosis and EBV Infection Case Definitions

EBV infection was defined by seroconversion to EBV non-vaccine antigens VCA (see below). Infectious mononucleosis was defined as a combination of symptoms of infectious mononucleosis AND seroconversion to EBV non-vaccine antigens. The following clinical symptoms of infectious mononucleosis were solicited upon suspicion of the disease: fatigue, sore throat, painful lymph nodes, fever (defined as >37.5° C.), sleeping too much, headache, sore muscles, nausea, sore joints, cough, rash. In addition, laboratory analyses were conducted on samples collected during a suspicion of infectious mononucleosis. The following abnormalities were considered as possible markers of the disease:
Increased ALT (out of normal range)
Increased AST (out of normal range)
Decreased Hb (out of normal range)
Increased White cell count (value >10000/mcl)
Increased lymphocytes (value >50%)
Bilirubinemia (bilirubin >upper limit of normal range)
Presence of Heterophile antibodies

Seroconversion to EBV non-vaccine antigens was defined as appearance of VCA-specific IgM or IgG in a subject who was initially seronegative.

For each suspicion of infectious mononucleosis, the available information on the suspicion (symptoms, laboratory and EBV serology data) was reviewed with an external EBV expert and during a Data Review Meeting before the database was locked (i.e. before breaking the study blind). Infectious mononucleosis was confirmed or not, in accordance with the definition mentioned above.

Classification of cases and non-cases was operationalised as follows:

A. Definite infectious mononucleosis cases corresponded to situations meeting the 3 following criteria:
1. two or more clinical symptoms (out of list specified above)
2. one or more laboratory abnormality (out of list specified above)
3. IgG (+/−IgM) seroconversion to EBV VCA AND no IgG seroconversion to CMV or toxoplasmosis;
   OR
IgM (& no IgG) seroconversion to EBV VCA AND no IgM no IgG seroconversion to CMV or toxoplasmosis
   B. Probable (likely) infectious mononucleosis cases corresponded to cases with a less clear-cut clinical presentation (or part of the data missing). This definition applied when the 2 following criteria were met:
1. subjects with no clinical symptoms reported but laboratory findings present, OR
   typical clinical symptoms but no laboratory abnormality reported
2. IgG (+/−IgM) seroconversion to EBV VCA AND no IgG seroconversion to CMV or toxo
   OR
   IgM (& no IgG) seroconversion to EBV VCA AND no IgM no IgG seroconversion to CMV or toxo
      C. Definite non-cases corresponded to situations where neither IgM nor IgG seroconversion to EBV VCA was observed.
      D. Possible cases were those situations where another infection was likely to have caused the clinical presentation. They required the following criteria:
IgM or IgG seroconversion to EBV VCA AND IgG seroconversion to CMV or toxo,
OR
IgM (& no IgG) seroconversion to EBV VCA AND IgM seroconversion to
CMV or toxo

### Laboratory Testing

All samples were analysed in a blinded fashion.

Hematology, biochemistry testing as well as evaluation of heterophile antibodies, cytomegalovirus and toxoplasmosis antibodies were conducted locally, by the laboratories of the study centers. All laboratories were accredited according to Belgian regulations for diagnostic testing of human samples.

Anti-VCA (EBV non-vaccine antigens) antibodies were tested centrally at Henogen s.a. The Biotest anti-VCA ELISA IgG and IgM assays were selected. Unlike other EBV serological tools, these assays are based on recombinant VCA antigens rather than lysates of infected cells. They were shown to not cross-react with vaccine-induced anti-gp350 antibodies. Anti-VCA immunofluorescence testing was also performed to confirm negative ELISA results at baseline (day 0), and to confirm positive ELISA results identified between month 1 and 19, as well as results lying in a grey-zone defined as the cut-off +/−20%.

### Results and Discussion:

Demographic data presented in Table 3 below, illustrate the conclusion that the 2 study groups were well balanced. Two of the 183 subjects enrolled in the trial were excluded from the intent-to-treat population as they left the study immediately after visit 1, and did not provide sufficient safety or immunogenicity data for analysis.

TABLE 3
Demographic data (intent to treat population)

| | Alum placebo | Gp350/Alum + 3D-MPL |
|---|---|---|
| N | 91 | 90 |
| Mean age (y) | 20.5 | 20.6 |
| Male gender (%) | 53.8 | 51.1 |
| Weight (kg) | 67.7 | 70.2 |
| Caucasian race (%) | 96.7 | 97.8 |

The drop-out rate in the trial remained very low, with a total of respectively 90 and 88 subjects (placebo and vaccine groups) remaining in the study at month 19.

To assess vaccine efficacy, the occurrence of both infectious mononucleosis and EBV infection was calculated in the 2 groups. Results are summarized in Table 4 below.

TABLE 4
Distributions of infectious mononucleosis and
EBV infection cases among vaccinees and placebo
recipients (intention-to-treat population)

| | Alum placebo | Gp350/Alum + 3D-MPL |
|---|---|---|
| N subjects | 91 | 90 |
| n IM cases | 9 | 2* |
| n asymptomatic infections | 9 | 11 |
| n infections (total) | 18 | 13 |

*difference between vaccine and placebo group reaches statistical significance

Of the 9 cases identified in the placebo group, one was classified as “probable”, because clinical symptoms were not reported adequately to the investigator. All 8 other cases met the definition of “definite cases”. The 2 vaccine failures were considered as definite case, although one of them was characterized by very limited disease symptoms, on the verge of our criteria for disease confirmation.

Of note, an additional case, which was reported before study unblinding but too late to be incorporated in the database, turned out also to be in a placebo recipient. When taking this additional case into account, the infectious mononucleosis case distribution becomes 10 in the placebo group vs. 2 in the vaccine group.

Overall the data indicate that the vaccine confers protection against infectious mononucleosis, while no protection against EBV infection could be demonstrated. The following statistical calculation supports the conclusion of efficacy against infectious mononucleosis. The odds to be a case is defined as Probability(case))/(1−Probability(case)). In the Placebo group the odds are (9/91)/(1−9/91)=0,11; similarly, the odds ratio in the gp350 group was 0.023.

The odds to be a case were 4.8 times as high in the placebo group as in the gp350 group, in other words the estimated odds ratio=4.8. An odds ratio of 1 means that the groups have equal probability of having cases. Statistical analysis tells us that in our study there is a 95% probability that the population odds ratio is greater than 1.30 (one sided confidence interval). This means that there are significantly more cases in the placebo group, applying a one sided test with α=0.05.

When stratifying for centre effects (calculation of a common odds ratio over centers), the results were as follows: estimated common odds ratio=5.10. Probability of population odds ratio being larger than 1.34 is 95%. Conclusion is the same as above.

This study was not designed to address the duration of protection. However, the FIGURE below, illustrating the time of occurrence of infectious mononucleosis in vaccinees and placebo recipients in the trial, suggests that the duration of protection upon vaccination with gp350/AS04 will exceed 2 years.

1. A method of preventing infectious mononucleosis in a subject comprising administering to the subject an immunogenic composition comprising an EBV gp350 membrane antigen or derivative thereof in combination with an adjuvant.

2. The method of claim 1, wherein the subject is at least 11 years of age.

3. The method of claim 1, wherein the gp350 is a homogeneous preparation of gp350.

4. The method of claim 1, wherein the EBV gp350 membrane antigen comprises a recombinant gp350 prepared from a non-splicing variant of the DNA expressing the gp350/220 proteins.

5. The method of claim 1, wherein the adjuvant comprises a mineral salt.

6. The method of claim 5, wherein the mineral salt is a member selected from the group consisting of aluminium hydroxide, aluminium phosphate and calcium phosphate.

7. The method of claim 5, wherein the adjuvant further comprises a non-toxic bacterial lipopolysaccharide derivative.

8. The method of claim 7, wherein the non-toxic bacterial lipopolysaccharide derivative is 3D-MPL.

9. The method of claim 7, wherein the mineral salt is aluminium hydroxide and the non-toxic bacterial lipopolysaccharide derivative is 3D-MPL.
